(19) United States
(12) Patent Application Publication
(10) Pub. No.: US 2023/0398095 A1
(43) Pub. Date: Dec. 14, 2023

(54) THERAPEUTIC AGENT FOR TREATMENT OF AGE-RELATED MACULAR DEGENERATION

(71) Applicants: Ahmad Alkayali, Temecula, CA (US); David Almeida, Temecula, CA (US); Eric Chin, Temecula, CA (US)

(72) Inventors: Ahmad Alkayali, Temecula, CA (US); David Almeida, Temecula, CA (US); Eric Chin, Temecula, CA (US)

(21) Appl. No.: 17/837,917

(22) Filed: Jun. 10, 2022

(51) Int. Cl. A61K 31/403; A61K 39/395; A61P 39/06

(52) U.S. Cl. CPC A61K 31/403; A61K 39/3955; A61P 39/06

(57) ABSTRACT

A method for treating age-related macular degeneration (AMD), either wet AMD or dry AMD. In the method, a therapeutic agent is administered to the patient. The therapeutic agent includes an antioxidant carbazole moiety fused to a nicotine analog. The patient is then monitored to determine the state of the age-related macular degeneration. The therapeutic agent may be administered orally, by injection, or by eye drops in preferred embodiments.

The present invention relates to disease treatments, particularly treatments of age-related macular degeneration.

### BACKGROUND OF THE INVENTION

Age-related macular degeneration (AMD) is the most common cause of decreased vision in the elderly population in the United States. Globally, it is one of the four most common causes of blindness after cataracts, preterm birth, and glaucoma. The number of people with AMD in 2020 was approximately 196 million, and this is expected to increase to 288 million in 2040. AMD causes damage to the macula, which is a small portion of the retina responsible for central visual acuity. This part of the retina is responsible for seeing fine details. Damage to the macula can prevent individuals from enjoying activities of daily living such as driving, reading, recognizing faces, or performing close work. Symptoms of AMD include dark areas or distortion of the central vision and permanent loss of central vision.

In some cases, AMD progresses slowly, and patients retain good vision without intervention; however, in other cases, it advances quickly and may lead to vision loss in one or both eyes. Unfortunately, many people are not aware that they have AMD until they have a noticeable vision complaint or until it is detected as an incidental finding during a routine eye examination. There are no definitive or reliable predictive markers for who will progress rapidly, who will respond well to treatment, or which family members of affected patients may be at greater risk for disease. Age is a significant risk factor for AMD, and the disease most commonly occurs after the age of 60. Other risk factors include smoking, ethnicity, and family history (genetics).

### Disease Classification and Current Available Treatment Options

AMD is typically classified either as dry or wet disease: the former (also known as “non-exudative AMD” or “atrophic AMD”) is most classically defined by yellow deposits under the retina (also known as drusen), and the latter by abnormal blood vessel growth leading to bleeding or fluid beneath the retina (also known as “choroidal neovascularization,” “exudation,” or “exudative AMD”). Approximately 10% of people with AMD have the wet form, which can sometimes cause more damage to the central vision than the dry form. Vision loss from wet AMD may be faster and more noticeable than the dry form. This classification between dry and wet disease is essential because the current treatment options are quite different. The earlier wet AMD is diagnosed and treated, the greater likelihood of preserving central vision. For dry macular degeneration, researchers at the National Eye Institute (NEI) found that daily intake of high-dose vitamins and minerals can slow the progression in people who have intermediate dry AMD or those who have more advanced dry AMD in one eye. More specifically, there has been shown a reduction in the risk of progression to more advanced AMD by 25% at five years. There are a number of manufacturers of these nutritional supplements, which are collectively sometimes labeled as “AREDS” or “AREDS2” vitamins based on the NEI-sponsored clinical trials “Age-Related Eye Disease Studies.” The clinically effective doses are as follows: 500 milligrams of vitamin C
400 international units of vitamin E
25 or 80 mg of zinc as zinc oxide
2 mg of copper as cupric oxide
15 mg beta-carotene OR 10 mg lutein and 2 mg zeaxanthin.

The most widely and effective treatment options for wet AMD patients involve multiple (often monthly) injections directly into the eye. Three agents are routinely used: bevacizumab (Avastin, off-label compounded product), ranibizumab (Lucentis, Genentech), and aflibercept (Eylea, Regeneron). Limitations to this therapy are the burden of monthly invasive treatment and the potential sequelae of atrophy or thinning of the retina. Additionally, there is sometimes a cohort of patients who do not respond to monthly therapy or have a subretinal hemorrhage, which often leads to irreversible scarring. Less common treatments for wet AMD include laser photocoagulation or photodynamic therapy.

### Clinical Unmet Needs & A Final Common Pathway

For dry AMD, in some cases, the retina eventually becomes thinner (or “atrophic”) and stops working correctly. Vision loss is usually gradual, but, in some cases, large areas of atrophy cause profound vision loss in the center portion of the sight. Unfortunately, eye vitamins do not reverse this damage, and there are no injections or surgeries to replace or restore the lost tissue. It is becoming increasingly apparent that the development of geographic atrophy (resulting in irreversible vision loss) may represent the final common pathway of all types of AMD. In 2004, the Eye Diseases Prevalence Group, using a meta-analysis of recent regional population-based studies in the United States, Australia, and Europe, estimated that late (advanced) AMD was present in more than 1.75 million individuals in the United States. The study projected that due to the longer survival of Americans, the number would increase to almost 3 million by 2020.

For wet AMD, a better anti-VEGF agent needs to be found that lasts longer than just one month and is more effective in improving vision and stopping disease progression. Potentially a better delivery system may be the solution to these unmet needs. Gene therapy, viral vectors, and encapsulated cell technology are some of these therapies currently being investigated in clinical trials

### Financial Considerations

### Dry AMD

The approximate cost of a box of AREDS2 vitamins (e.g., Bausch and Lomb PreserVision AREDS2 vitamins, 120 soft gels, twice daily; two-month supply) is approximately $30. Once indicated, this therapy is needed indefinitely. Amsler grid monitoring is readily available via readily available and distributable online materials. Smoking cessation should always be encouraged since this is a known risk factor for macular degeneration progression. A pack of cigarettes averages between six to eight dollars per pack. In some states, the cost of cigarettes can be as high as $12.85 per pack (New York). There is the tremendous market potential for a therapeutic drug with efficacy in treating dry AMD and preventing exudative AMD that—given the cost of treatment for exudative AMD (see below)—would have beneficial cost-benefit implications.

### Neovascular AMD

The approximate cost per injection for bevacizumab is in the range of $100 to $250. The price per injection (per patient per month) for ranibizumab and aflibercept is around $2000. One study reported the average drug cost of bevacizumab, ranibizumab, and aflibercept over six months to be $326, $11,400, and $9,720, respectively. In 2010, Medicare expenditure for intravitreal injection was approximately $200 million. In 2013, the General Accounting Office released a report on Part B drug costs for 2010 and found that ranibizumab was the third-highest expenditure for Medicare beneficiaries at $1.18 billion. Similarly, there is significant market potential in the wet AMD treatment therapeutics landscape to dry AMD. Our minor molecule candidate is postulated to be directly positioned for the treatment of dry AMD and possibly as a synergy treatment with vascular endothelial growth factor antagonism in wet AMD.

### Oxidative Stress

Oxidative stress is cellular damage caused by reactive oxygen species (ROS) and is implicated in many age-related disorders like AMD. ROS include free radicals, hydrogen peroxide, and singlet oxygen which are often the byproducts of oxygen metabolism. The retina is particularly susceptible to oxidative stress because of its high oxygen consumption, its high proportion of polyunsaturated fatty acids, and its exposure to visible light. 15 Moreover, photochemical retinal injury is attributable to oxidative stress, and the antioxidant vitamins (A, C, E) protect against this type of injury. There is also strong evidence suggesting that lipofuscin is derived from oxidatively damaged photoreceptor outer segments and is a photoreactive substance. Macular pigments and antioxidants limit retinal oxidative damage by absorbing blue light and neutralizing ROS. The concept that dry AMD can be attributed to cumulative oxidative stress is supported by the beneficial treatment effect of antioxidant nutraceuticals observed in age-related eye disease studies (AREDS).

### Angiogenesis

Angiogenesis is a complex multifactorial process regulated by a balance between pro- and anti-angiogenic molecules. Angiogenic stimuli (e.g., hypoxia or inflammatory cytokines) induce the expression and release of angiogenic growth factors such as vascular endothelial growth factor (VEGF). These growth factors stimulate endothelial cells in the existing vasculature to proliferate and migrate through the tissue to form new endothelialized channels. The binding of VEGF is the gold standard treatment for wet (exudative or neovascular) AMD.

### Human Tenon's Capsule Fibroblasts (HTCF)

Human Tenon's capsule fibroblasts (HTCF) reside beneath the conjunctiva in the Tenon's capsule (TC), which functions as a fibrous connective tissue that fuses with the sclera anteriorly and extends back to the meninges of the optic nerve posteriorly.
Fibroblast cells impact ocular disease across many pathologies.
Fibrosis results from the aberrant response of fibroblasts to stress in AMD, glaucoma, and proliferative vitreoretinopathy.
Oxidative stress in AMD activates fibroblasts and promotes subretinal scarring; this is a significant sequela of AMD pathology.
Fibrosis in the subconjunctival space is the primary cause of surgical failure in glaucoma.
HTCFs have several features which allow them to be ideal in the investigation of the potential therapeutic effects of novel wound modulating agents. Primary human cultures can be generated from tissue available at the time of ocular Surgery and HTCFs can be investigated in two- and three-dimensional microfluidic in vitro models.
HTCFs have been well characterized in the Hutnik laboratory, with cell lines established from a number of different donors.

What is needed is a treatment for AMD that addresses both oxidative stress and angiogenesis.

### SUMMARY OF THE INVENTION

The present invention provides a method for treating age-related macular degeneration (AMD), either wet AMD or dry AMD. In the method, a therapeutic agent is administered to the patient. The therapeutic agent includes an antioxidant carbazole moiety fused to a nicotine analog. The patient is then monitored to determine the state of the age-related macular degeneration. The therapeutic agent may be administered orally, by injection, or by eye drops.

### BRIEF DESCRIPTION OF THE DRAWINGS

FIG. 1 shows the chemical structure of carbazole.

FIG. 2 shows the chemical structure of nicotine.

FIG. 3 shows a preferred therapeutic agent for the treatment of age-related macular degeneration.

FIGS. 4-12 show experimental results.

### DETAILED DESCRIPTION OF THE PREFERRED EMBODIMENTS

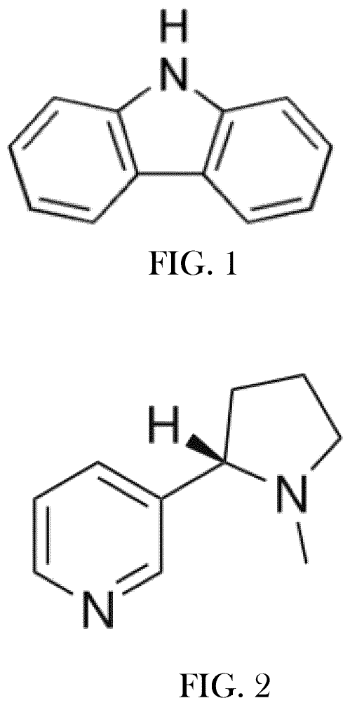

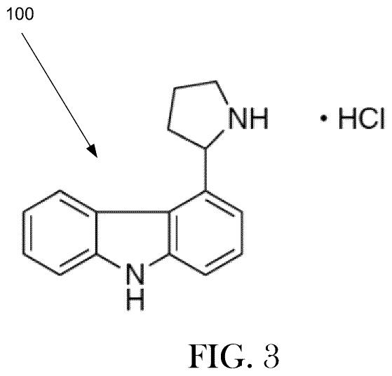

FIG. 3 shows therapeutic agent 100. Therapeutic agent 100 is best characterized as a vascular disruptive agent and includes an antioxidant carbazole moiety (FIG. 1) fused to a nicotine analog (FIG. 2). The carbazole moiety is a potent antioxidant ideal for oxidative stress encountered in dry AMD. The nicotine analog interferes non-selectively with the nicotinic acetylcholine angiogenic pathways and may have utility in both dry and wet forms of AMD.

FIG. 1 shows the chemical structure of carbazole. Carbazole is an aromatic heterocyclic organic compound. It has a tricyclic structure consisting of two six-membered benzene rings fused on either side of a five-membered nitrogen-containing ring.

Carbazole is a potent antioxidant pharmacophore that has been analyzed by Applicant extensively. Its use as part of cardiovascular drugs is attributable to the significant antioxidant effect of carbazole and its ability to neutralize reactive oxygen species (ROS) by donating electron density from the ring structure. Carbazole is helpful as an antioxidant structure in treating macular diseases attributed to significant oxidative stress. Through in silico experimentation, Applicant has shown that the carbazole fragment can neutralize oxidant stress by donating electron density from its aromatic heterocyclic ring structure.

FIG. 2 shows the chemical structure of nicotine. Nicotine is a parasympathomimetic alkaloid found in the nightshade family of plants (Solanaceae). Nicotine has angiogenic effects mediated through non-neuronal nicotinic acetylcholine receptors and increasing concentrations of nonselective antagonism completely and reversibly inhibited endothelial network formation. Non-specific nicotinic antagonists have been shown to inhibit endothelial cell proliferation and blood vessel formation. Nicotinic acetylcholine receptors are ligand-gated cation channels and consist of pentameric complexes. In the neurosensory retina, nicotinic acetylcholine receptor subunits are expressed and localized to diverse retinal neurons and microglia; consequently, pharmacological processes interact with these nicotinic receptors have the potential for a myriad of significant benefits. Antagonism of non-neuronal nicotinic acetylcholine receptors plays an essential role in physiological and pathological angiogenesis and is a novel avenue for therapeutic modulation of angiogenesis relevant to exudative forms of AMD.

FIG. 3 shows the chemical structure of the preferred therapeutic agent 100. Therapeutic agent 100 includes the presence of two distinct pharmacophores consisting of the antioxidant carbazole moiety and a nicotine analog. Therapeutic potential as an antioxidant (carbazole moiety) and vascular disruptive agent (nicotinic effects) hold promise for multiple therapeutic targets, including dry AMD, exudative neovascular AMD, macular ischemia, and diabetic retinopathy.

Applicant has designed a novel oral small-molecule therapeutic agent, therapeutic agent 100, which is constructed of two distinct pharmacophores: an antioxidant carbazole moiety that is fused to a nicotine analog. The carbazole moiety is a potent antioxidant ideal for the oxidative stress encountered in dry AMD. The ability to mitigate the oxidant stress experienced in the macula by the carbazole pharmacophore is novel, given the overall capacity of this pharmacophore compared to existing therapeutic options.

Moreover, the nicotine analog is designed to interfere non-selectively with the nicotinic acetylcholine angiogenic pathways and may have utility in both dry and wet forms of AMD. The proposed mechanism is best described as a “vascular disruptive agent” (VDA). VDA agents can stabilize retinal endothelial cells and modulate the aberrant angiogenic mechanisms beyond simple VEGF blockade. Herein, the preferred therapeutic agent 100 can treat anomalous scarring diseases such as glaucoma and proliferative vitreoretinopathy.

This multi-pharmacophore therapeutic is novel in the ophthalmic space and is an extension of the pharmacodynamics concept of the “combined drug mechanism of action” standard in the oncology and chemotherapy literature, which shows that certain synergistic drug combinations may act as a more potent version of a single drug. By having two active pharmacophores present, therapeutic agent 100 has been found to be a viable therapeutic agent with two possible mechanisms of action. A primary limitation of current exudative anti-VEGF therapies is the ongoing development of macular atrophy; however, therapeutic agent 100 provides an oral agent that both prevents macular atrophy via an antioxidant mechanism while at the same time stabilizes retinal vascular networks via the VDA mechanism.

### Usages of the Preferred Therapeutic Agent

### 1) Prevention of Dry AMD Progression (Antioxidant Mechanism)

Therapeutic agent 100 is used as a neuroprotective compound in patients with mild dry AMD, to prevent the progression to moderate dry AMD and advanced geographic atrophy.

### 2) Treatment of Dry AMD (Antioxidant Mechanism)

Therapeutic agent 100 is used to delay geographic atrophy growth in patients with moderate and high-risk dry AMD. Rate of growth of the atrophic lesion to end-stage or advanced dry AMD as a measure of progression of the disease.

### 3) Prevention of Exudative AMD (Antioxidant and Vascular Disruptive Mechanism)

Therapeutic agent 100 is used in preventing wet AMD in patients with moderate and high-risk dry AMD. Via the vascular disruptive agent (VDA) mechanism of therapeutic agent 100, potential prevention of wet AMD in a patient who has not yet developed choroidal neovascularization.

### 4) Treatment of Exudative AMD (Antioxidant, Vascular Disruptive, and Anti-Angiogenic Mechanisms)

Therapeutic agent 100 is used as a synergy to the current anti-VEGF standard of care. Concurrent treatment of therapeutic agent 100 with an anti-VEGF agent in patients with exudative AMD to assess visual acuity and treatment burden outcomes. The novel potential of antioxidant and VDA actions in wet AMD.
Therapeutic agent 100 is used as an oral agent to reduce the burden of monthly anti-VEGF injections.

### 5) Treatment of Macular Ischemia (VDA Mechanism)

Therapeutic potential of therapeutic agent 100 in macular ischemia via endothelial cell stabilization owing to the effects of a combined carbazole/antioxidant and nicotine/VDA mechanisms.

### 6) Treatment of Diabetic Retinopathy (VDA Mechanism)

Therapeutic agent 100 is used as a VDA to improve vascular status in diabetic retinopathy. Moreover, potential application for other macular diseases with oxidant and ischemic pathophysiology, such as macular edema from retinal vein occlusion, is also possible.

### Mode of Delivery

In the preferred embodiment, therapeutic agent 100 may be administered to the patient orally, by injection, or via eye drops.

### Experiments

Experiments were conducted to determine the effectiveness of therapeutic agent 100. It was hypothesized that therapeutic agent 100 would have an effect on modulating stress-induced ocular fibroblasts. It was predicted that therapeutic agent 100 would salvage ROS-impaired cellular metabolic activity and reduce cell cytotoxicity.

### Oxidation Stressor

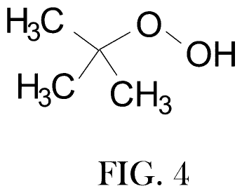

Tert-butyl hydroperoxide (t-BOOH) (FIG. 4) was utilized as a chemical oxidant to induce oxidative stress (e.g., ROS generation) in cells to model the situation that occurs in AMD. The goal was to use t-BOOH to achieve an approximate 40% reduction in cell metabolic activity.

### Experimental Method

The following preferred method was utilized in conducting the experiment:
1) Culture cells (90% confluence)
2) Load 5×10⁴ cells/well in 24-well plates
3) Change to serum-free medium overnight
4) Treat cells
5) Measure cell metabolic activity (MTT) or % cytotoxicity (LDH)
6) Normalize results
7) Replicate experiment with cell lines from different patients
8) Conduct statistical analysis

### Further Detail on Experimental Methods

### Oxidative Stressor Dosage Curve

1. Treat cells with serum free medium and tert-butyl hydroperoxide (t-BOOH) at 0 mM (VC), mM, 0.1 mM, 0.5 mM, 1 mM and 2 mM for 30 min, 1 h, 2 h, 3 h

2. Measure cell metabolic activity with 3-(4,5-dimethylthiazol-2-yl)-2,5-diphenyltetrazolium bromide (MTT) assay

3. Measure % cytotoxicity with LDH assay

### Therapeutic Agent 100 Dosage Curve

1. Create a stock solution of 0.1 M of therapeutic agent 100 by dissolving therapeutic agent 100 in nonpolar solvent dimethyl sulfoxide (DMSO)

2. Treat cells with serum-free medium and therapeutic agent 100 at 011M (VC), 10011M DMSO, 10011M CTX1, 1011M DMSO, 1011M CTX1, 111M CTX1 for 1 h, 3 h, overnight, 24 h

3. Measure cell metabolic activity with MTT assay

### Oxidative Stressor and Therapeutic Agent 100

Combine different combinations of t-BOOH and therapeutic agent 100 to assess effects on cell metabolic activity.

### Results

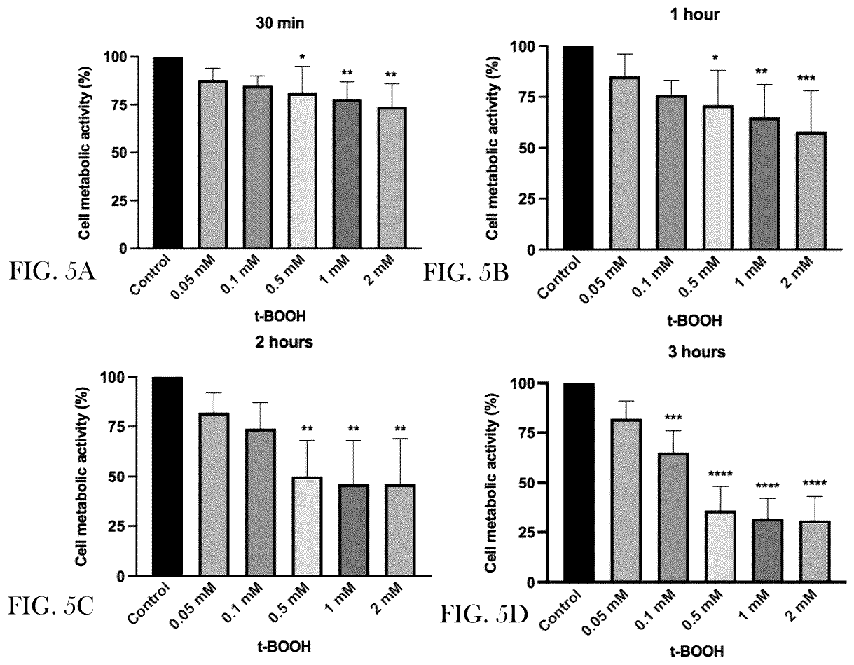

FIGS. 5A-5D show the cell metabolic activity of HTCF cells in response to treatment with tert-butyl hydroperoxide (t-BOOH). Dosedependent effect is shown of a: 30 min incubation (FIG. 5A), 1 h incubation (FIG. 5B), 2 h incubation (FIG. 5C), and 3 h incubation (FIG. 5D) with t-BOOH. *Significantly different from the control sample (p<0.05; n=4), ** (p<0.01), *** (p<0.001).

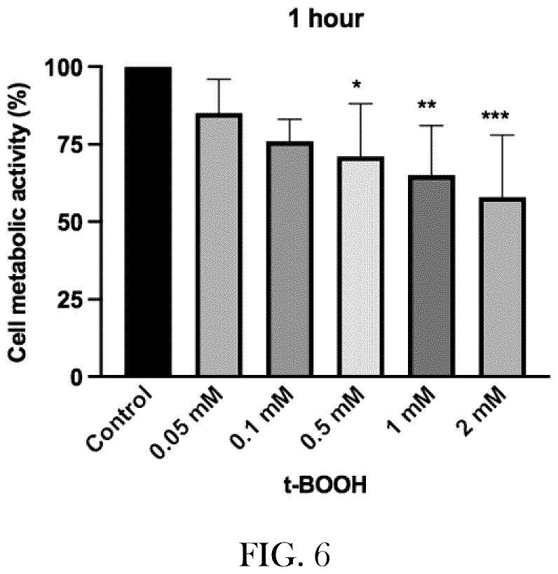

FIG. 6 shows the cell metabolic activity of HTCF cells in response to treatment with tert-butyl hydroperoxide (t-BOOH). Dose-dependent effect of a one-hour incubation. Note the significant effect versus the control sample (p<0.05; n=4), ** (p<0.01), ***(p<0.001).

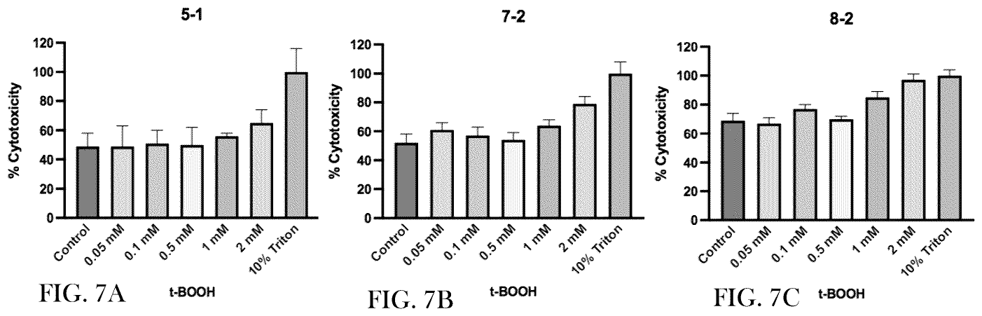

FIGS. 7A-7C shows the percent cytotoxicity of HTCF cells in response to treatment with tert-butyl hydroperoxide (t-BOOH). Dose-dependent effect of a one-hour incubation with t-BOOH on three separate cell lines.

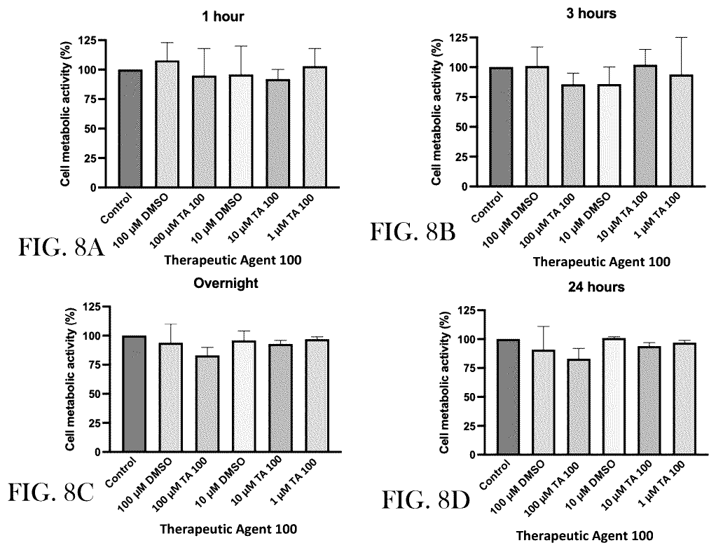

FIGS. 8A-8D shows the cell metabolic activity of HTCF cells in response to treatment with therapeutic agent 100 (shown in FIG. 8 also as TA 100). The dose-dependent effect is shown of one-hour incubation (FIG. 8A), two-hour incubation (FIG. 8B), and overnight incubation (FIG. 8C), 24 hours incubation (FIG. 8D) with therapeutic agent 100.

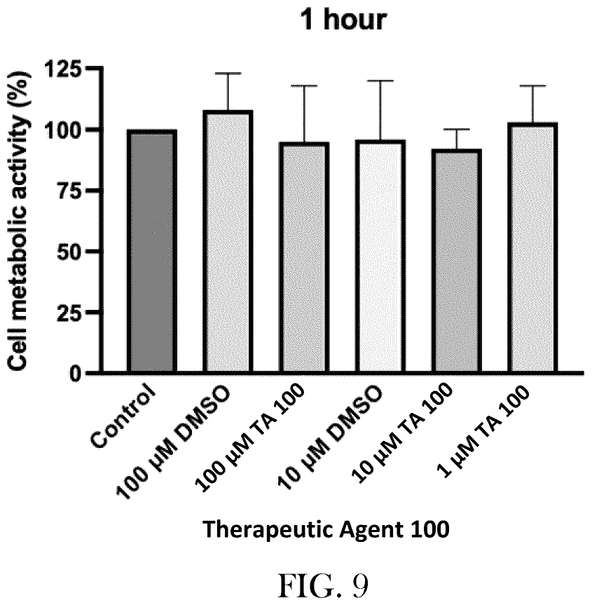

FIG. 9 shows the cell metabolic activity of HTCF cells in response to treatment with therapeutic agent 100. Dose-dependent effect of a one-hour incubation.

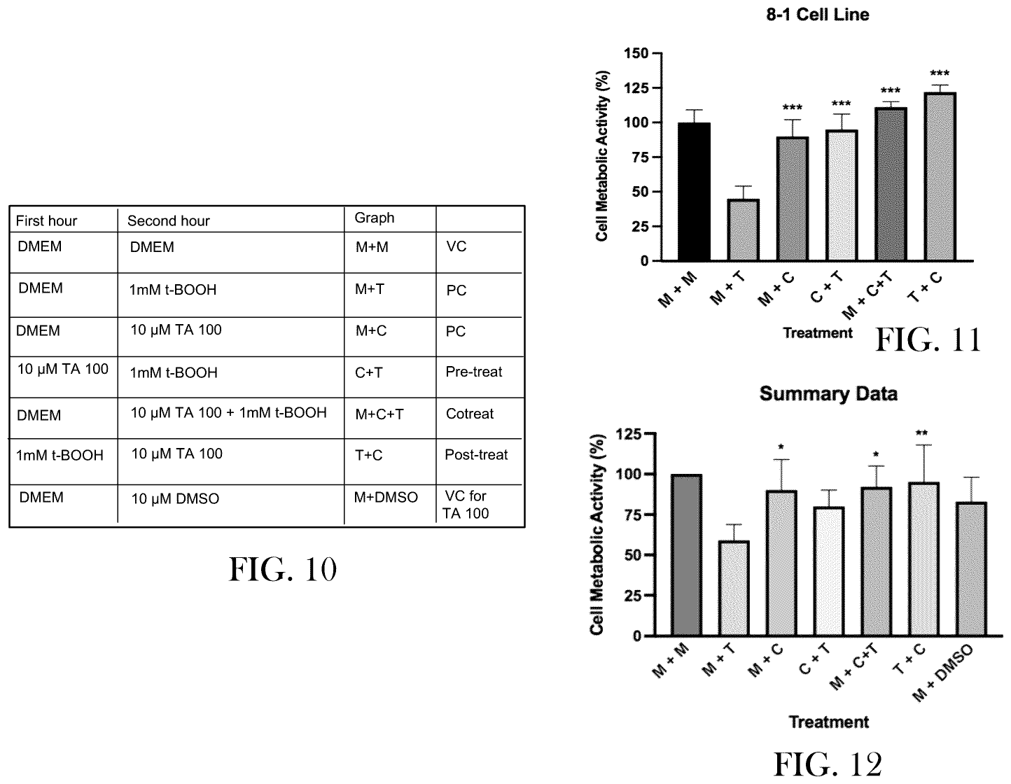

FIGS. 10-12 shows the cell metabolic activity of HTCF cells in response to different treatments for one cell line (FIG. 11) and all cell lines tested (FIG. 12). *Significantly different from the positive control sample of t-BOOH (p<0.05; n=5), ** (p<0.01), *** (p<0.001).

### CONCLUSION

Based on the above experimental results, there is strong evidence that therapeutic agent 100 reverses the reduction in cellular metabolic activity induced by t-BOOH in HTCF. Also, it is noted that co-treatment and post-treatment strategies demonstrate significant differences from the positive control. Additionally, therapeutic agent 100 appears to salvage the oxidative stress induced by t-BOOH in HTCF and reduce fibroblast cell proliferation.

Although the above preferred embodiments have been described with specificity, persons skilled in this art will recognize that many changes to the specific embodiments disclosed above could be made without departing from the spirit of the invention. Therefore, the attached claims and their legal equivalents should determine the scope of the invention.

What is claimed is:

1) A method of treating age-related macular degeneration in a mammal said method comprising the steps of:
a. administering to said mammal a therapeutic agent comprising an antioxidant carbazole moiety fused to a nicotine analog, and
b. monitoring said mammal to determine the state of said age-related macular degeneration.

2) The method as in claim 1, wherein said age-related macular degeneration is wet disease age-related macular degeneration.

3) The method as in claim 1, wherein said age-related macular degeneration is dry disease age-related macular degeneration.

4) The method as in claim 1, wherein said therapeutic agent is administered orally.

5) The method as in claim 1, wherein said therapeutic agent is administered by injection.

6) The method as in claim 1, wherein said therapeutic agent is administered via eye drops.

7) The method as in claim 1, wherein said mammal is a human.

8) The method as in claim 1, wherein said therapeutic agent is used as a neuroprotective compound, in patients with mild dry age-related macular degeneration, in preventing the progression to moderate dry age-related macular degeneration and advanced geographic atrophy.

9) The method as in claim 1, wherein said therapeutic agent is used in delaying the growth of geographic atrophy in patients with moderate and high-risk dry age-related macular degeneration.

10) The method as in claim 1, wherein said therapeutic agent is used in preventing wet age-related macular degeneration in patients with moderate and high-risk dry age-related macular degeneration.

11) The method as in claim 1, wherein said therapeutic agent is used as synergy treatment to current anti-VEGF standard of care.

12) The method as in claim 1, wherein said therapeutic agent is used as an oral agent to reduce the burden of monthly anti-VEGF injections.

13) The method as in claim 1, wherein said therapeutic agent is used as a treatment of macular ischemia.

14) The method as in claim 1, wherein said therapeutic agent is used as a vascular disruptive agent to improve vascular status in diabetic retinopathy.
